(19) United States
(12) Patent Application Publication
(10) Pub. No.: US 2018/0001019 A1
(43) Pub. Date: Jan. 4, 2018

(54) PRE-FILLED DRUG CARTRIDGE WITH STOPPER PARTIALLY PROTRUDING FROM IT FOR FACILITATING PRIMING

(71) Applicant: SANOFI-AVENTIS DEUTSCHLAND GMBH, Frankfurt am Main (DE)

(72) Inventors: Michael Schabbach, Frankfurt am Main (DE); Daniel Auernhammer, Frankfurt am Main (DE)

(21) Appl. No.: 15/542,568

(22) PCT Filed: Jan. 11, 2016
(86) PCT No.: PCT/EP2016/050339

§ 371 Date: Jul. 10, 2017

(30) Foreign Application Priority Data
Jan. 12, 2015 (EP) 15150744.9

(51) Int. Cl. A61M 5/158; A61J 1/20; A61M 5/14

(52) U.S. Cl. CPC A61M 5/158; A61J 1/2041; A61M 2005/1402

(57) ABSTRACT

Disclosed is a combination of an infusion set consisting of infusion tubing and needle connected to a drug cartridge, the latter comprising a cylindrical body of inner diameter and having an open rear end, wherein a stopper is slidably disposed within the body. In an initial state after the cartridge has been filled, the stopper is partially disposed within the body such that the stopper protrudes from the rear end of the body by a distance. Pushing the stopper into the cartridge by said distance, i.e. until the stopper is flush with the rear end, displaces a predetermined volume according to E, B and the cylindrical geometry of the cartridge, which is large enough to effect complete air priming of the cartridge, infusion tubing and infusion needle.

### CROSS-REFERENCE TO RELATED APPLICATIONS

This application is a U.S. national stage application under 35 USC §371 of International Application No. PCT/EP2016/050339, filed on Jan. 11, 2016, which claims priority to European Patent Application No. 15150744.9, filed on Jan. 12, 2015, the entire contents of which are incorporated herein by reference.

### TECHNICAL FIELD

The disclosure relates to a drug cartridge.

### BACKGROUND

When preparing an injection of a drug, e. g. by pump, a user may have to connect a tubing with a cannula to a drug cartridge. Before the tubing can be connected to an injection site such as a patient's skin, a priming shot has to be performed, i. e. a volume of the drug is displaced from the cartridge for filling the whole tubing until drug starts leaking out of the end of the tubing such that residual air is displaced from the tubing. The priming may be performed by the pump or by manually pushing a stopper within the cartridge before inserting the cartridge into the pump. As the stopper may be hard to access, the user may be tempted to use objects such as a ball pen to displace the stopper. This may result in excess displacement of drug or damage to the cartridge.

### SUMMARY

In some embodiments, a drug cartridge comprises a body having an open rear end, wherein a stopper is slidably disposed within the body, wherein in an initial state the stopper is partially disposed within the body such that the stopper protrudes from the rear end of the body.

In order to prime the cartridge, the rear end of the cartridge with the protruding stopper may be pushed against a surface, e.g. a desk. A break-loose force between the stopper and an inner wall of the cylindrical body is thus overcome, i.e. the cartridge is primed, and the stopper is moved within the body until the stopper is flush with the rear end. If the cartridge is then inserted into a drug delivery device, the stopper may be moved much easier as the break-loose force increases over a long storage time. This allows for specifying a pump or drive mechanism of the drug delivery device to provide a force lower than the break-loose force.

In an exemplary embodiment, the body has a closed front wall with an opening to which a tubing can be removably fitted.

In an exemplary embodiment, an end of the tubing opposite the one fitted to the opening of the closed front wall is equipped with a cannula.

In an exemplary embodiment, the cylindrical body comprises or consists of glass.

In another exemplary embodiment, the cylindrical body comprises or consists of plastics.

In an exemplary embodiment, the stopper protrudes from the rear end of the body by an extension such that when the cartridge is being pushed against a surface until the rear end abuts the surface, the stopper is moved within the body by such a distance that a predetermined volume of medicament is displaced from the cartridge thereby displacing air from the tubing and from the cannula, if applicable.

In an exemplary embodiment, the predetermined volume is at least as large as a volume of the tubing.

In an exemplary embodiment, the predetermined volume substantially equals a volume of the tubing and the cannula.

Thus, a user is enabled to prime the drug cartridge without having to resort to additional tools. Other than such tools, the peg on the packaging cannot get lost. The priming with the arrangement can be performed much faster than with a pump of a drug delivery device. Other than with additional tools excess displacement of medicament or damage to the cartridge, e.g. a glass cartridge, is avoided.

In an exemplary embodiment, the body has an internal diameter B, wherein the movement distance D of the stopper required to displace the predetermined volume V is given by the equation

$$D = \frac{4 V}{π {B}^{2}} ,$$

wherein the extension of the stopper from the rear end is substantially equal to the movement distance D.

In an exemplary embodiment, the extension of the stopper can be slightly larger than the movement D in order to account for filling tolerances of the cartridges which may result in varying initial positions of the stopper in different cartridges.

In an exemplary embodiment, the predetermined volume is 1 ml.

Further scope of applicability of some embodiments will become apparent from the detailed description given hereinafter. However, it should be understood that the detailed description and specific examples, while indicating exemplary embodiments of the invention, are given by way of illustration only, since various changes and modifications within the spirit and scope of the invention will become apparent to those skilled in the art from this detailed description.

Certain aspects will become more fully understood from the detailed description given herein below and the accompanying drawings which are given by way of illustration only, and thus do not limit the present invention.

### BRIEF DESCRIPTION OF THE FIGURES

FIG. 1 is a schematic view of an exemplary embodiment of a drug cartridge prior to priming, and

FIG. 2 is a schematic view of the drug cartridge after priming.

Corresponding parts are marked with the same reference symbols in all figures.

### DETAILED DESCRIPTION

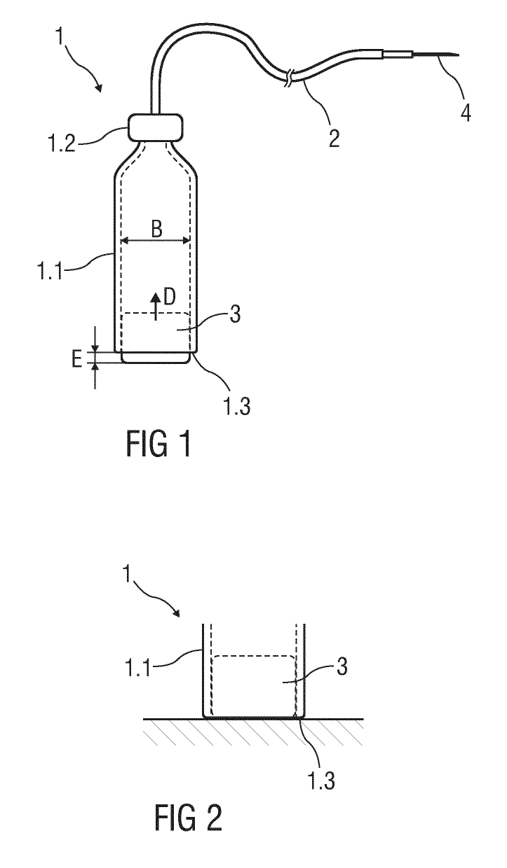

FIG. 1 is a schematic view of an exemplary embodiment of a drug cartridge 1 prior to priming.

The cartridge 1 comprises a cylindrical body 1.1 having a closed front wall 1.2 with an opening or septum to which a tubing 2 can be removably fitted and an open rear end 1.3. A stopper 3 is slidably disposed within the cylindrical body 1.1. A cavity for storing a medicament, e.g. insulin, is thus defined within the cylindrical body 1.1 between the closed front wall 1.2 and the stopper 3. The stopper 3 fluid tightly seals this cavity and displaces the medicament from the cavity when being moved towards the closed front wall 1.2. In an exemplary embodiment, an end of the tubing 2 opposite the one fitted to the opening of the closed front wall 1.2 is equipped with a cannula 4 or other infusion member adapted to be inserted into an injection site such as a user's skin.

In an exemplary embodiment, the cylindrical body 1.1 comprises or consists of glass. In other embodiments, the cylindrical body 1.1 may comprise or consist of plastics or another suitable material.

In an exemplary embodiment, in an initial state, the stopper 3 of the pre-filled cartridge 1 is partially disposed within the body 1.1 such that the stopper 3 protrudes from the rear end 1.3 of the body 1.1 by an extension E.

In order to prime the cartridge 1, the rear end 1.3 of the cartridge 1 with the protruding stopper 3 may be pushed against a surface, e.g. a desk. A break-loose force between the stopper 3 and an inner wall of the cylindrical body 1.1 is thus overcome, i.e. the cartridge 1 is primed, and the stopper 3 is moved within the body 1.1 until the stopper 3 is flush with the rear end. FIG. 2 is a schematic view of the drug cartridge 1 after priming. If the cartridge 1 is then inserted into a drug delivery device (not illustrated) the stopper 3 may be moved much easier as the break-loose force increases over a long storage time.

In an exemplary embodiment, the extension E of the stopper 3 is such that when the cartridge 1 is being pushed against the surface until the rear end 1.3 abuts the surface, the stopper 3 is moved within the body 1.1 towards the front wall 1.2 by such a distance that a predetermined volume V of medicament is displaced from the cartridge 1 sufficient to fill the tubing 2 and the cannula 4, which a user may have attached to the cartridge 1 prior to this, thereby displacing air from the tubing 2 and the cannula 4.

The volume V of medicament to be displaced for priming depends on the internal volume of the tubing 2 and the cannula 4. A movement D of the stopper 2 required to displace this volume V is given by

$$D = \frac{4 V}{π {B}^{2}} ,$$

wherein B is an internal diameter of the body 1.1 of the cartridge 1. The extension E of the stopper 3 may be such that a somewhat larger movement D of the stopper 3 is caused in order to account for filling tolerances of different cartridges 1 which may result in varying initial positions of the stopper 3 in different cartridges 1.

In an exemplary embodiment, the predetermined volume V of medicament to be displaced for priming may be 1 ml.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser- Ser-Gly-Ala-Pro-Pro-Pro-Ser-NH2.

Exendin-4 derivatives are, for example, selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28]Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28]Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28]Exendin-4(1-39),

des Pro36 [Trp(O2)25, Asp28] Exendin-4(1-39),

des Pro36 [Trp(O2)25, IsoAsp28]Exendin-4(1-39),

des Pro36 [Met(O)14 Trp(O2)25, Asp28] Exendin-4(1-39),

des Pro36 [Met(O)14 Trp(O2)25, IsoAsp28]Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(G1u)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra-low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds, which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and E have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three on the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

Those of skill in the art will understand that modifications (additions and/or removals) of various components of the apparatuses, methods and/or systems and embodiments described herein may be made without departing from the full scope and spirit of the present invention, which encompass such modifications and any and all equivalents thereof.

### LIST OF REFERENCES

1 cartridge
1.1 body
1.2 front wall
1.3 rear end
2 tubing
3 stopper
4 cannula
B internal diameter
D movement
E extension
V volume

1-11. (canceled)

12. A drug cartridge, comprising a body having an open rear end, wherein a stopper is slidably disposed within the body, wherein in an initial state the stopper is partially disposed within the body such that the stopper protrudes from the rear end of the body.

13. The drug cartridge according to claim 12, wherein the body has a closed front wall with an opening to which a tubing can be removably fitted.

14. The drug cartridge according to claim 13, wherein the tubing has an end fitted to the opening of the closed front wall, and an end of the tubing opposite the end fitted to the opening of the closed front wall is equipped with a cannula.

15. The drug cartridge according to claim 12, wherein the cylindrical body comprises glass.

16. The drug cartridge according to claim 12, wherein the cylindrical body comprises plastics.

17. The drug cartridge according to claim 12, wherein the stopper protrudes from the rear end of the body by an extension such that when the cartridge is being pushed against a surface until the rear end abuts the surface, the stopper is moved within the body by such a movement distance that a predetermined volume of medicament is displaced from the cartridge.

18. The drug cartridge according to claim 17, wherein the predetermined volume is at least as large as a volume of the tubing.

19. The drug cartridge according to claim 17, wherein the predetermined volume substantially equals a volume of the tubing and a cannula connected to the tubing.

20. The drug cartridge according to claim 17, wherein the body has an internal diameter, wherein the movement distance of the stopper required to displace the predetermined volume is given by the equation
$$D = \frac{4 V}{π {B}^{2}} ,$$
wherein the extension is substantially equal to the movement distance.

21. The drug cartridge according to claim 17, wherein the body has an internal diameter, wherein the movement distance of the stopper required to displace the predetermined volume is given by the equation
$$D = \frac{4 V}{π {B}^{2}} ,$$
wherein the extension is slightly larger than the movement distance.

22. The drug cartridge according to claim 17, wherein the predetermined volume is 1 ml.

23. The drug cartridge according to claim 12, wherein the drug cartridge contains medicament.

24. A method for priming a drug cartridge comprising, a body having an open rear end, wherein a stopper is slidably disposed within the body, wherein in an initial state the stopper is partially disposed within the body such that the stopper protrudes from the rear end of the body, the method comprising:
orienting the drug cartridge such that the rear end of the body is facing a surface; and
applying a force to the drug cartridge towards the surface such that the protruded stopper moves into the body of the drug cartridge and the outward facing surface of the protruded stopper becomes flush with the outward facing edge of the body of the drug cartridge.

25. The method of claim 24, wherein the surface is flat.

26. The method of claim 24, further comprising removably connecting tubing to the drug cartridge prior to applying the force to the drug cartridge.
